(19) United States
(12) Patent Application Publication
(10) Pub. No.: US 2024/0052432 A1
(43) Pub. Date: Feb. 15, 2024

(54) Methods of Detecting Listeria from an Environmental Sample

(71) Applicant: Neogen Corporation, Lansing, MI (US)

(72) Inventors: Preetha K. BISWAS, Okemos, MI (US); Lei ZHANG, Okemos, MI (US); Lin Li WALKER, East Lansing, MI (US); Robert S. Donofrio, Williamston, MI (US)

(21) Appl. No.: 18/249,590

(22) PCT Filed: Oct. 21, 2021
(86) PCT No.: PCT/US21/55962

§ 371 Date: Apr. 19, 2023

Related U.S. Application Data
(60) Provisional application No. 63/094,945, filed on Oct. 22, 2020.

(51) Int. Cl. C12Q 1/689; C12Q 1/24

(52) U.S. Cl. CPC C12Q 1/689; C12Q 1/24

(57) ABSTRACT

Detection of Listeria from an environmental sample is described herein. In particular, the detection can be completed with results available the same day or same work shift, e.g., in about 12 hours or less, or about 6 to 10 hours. The Same Day detection utilizes a short incubation period and/or concentration steps coupled with a rapid detection assay to produce same day results.

### CROSS-REFERENCE TO RELATED APPLICATION

This application claims priority to and benefit of U.S. provisional application No. 63/094,945, filed Oct. 22, 2020, the entire contents of which are incorporated herein by reference.

### TECHNICAL FIELD

The present invention relates to the detection of Listeria from an environmental sample. In particular, detection from an environmental sample can be completed in a single day or single work shift.

### BACKGROUND

Listeria species are very common and can be found almost anywhere in the environment. As indicator organisms, Listeria spp. are useful in assessing the potential presence of the pathogenic Listeria monocytogenes in the food processing plant environment. Listeria monocytogenes is a harmful pathogen that can be especially problematic in the food industry as it is associated with human foodborne listeriosis. There is a need for methods and diagnostic tools that rapidly detect and identify Listeria in environmental samples, including environmental samples for food safety testing. The devices, methods, and kits of the present invention satisfy this and other needs.

### SUMMARY

The present invention provides users with a means to assay for the presence of Listeria in environmental samples from food production, food processing or food service sites, wherein the results are obtained on the same day or same work shift, e.g., in about 12 hours or less, or about 6 to 10 hours. Conventional methods for assaying for Listeria from environmental samples require long incubation periods of 16 to 24 hours or more and thus results are not available on the same day, or at least not on the same work shift. The same day results, as described herein, provide advantages for the food industry, for example, by allowing detection of Listeria before food products leave a facility.

In one aspect, provided herein is a method of detecting Listeria in an environmental sample. In some embodiments, the sample is from a low-concentration environment. The method includes the steps of: collecting an environmental sample comprising cells using a collection device; transferring the sample to an incubation container containing about 50 ml or less of enrichment media; incubating the cells in the incubation container for from about 12 hours or less; lysing the cells to release nucleic acids from the cells; and performing a detection assay that targets nucleic acid sequences for amplification and detection of Listeria nucleic acids released from the cells.

Embodiments of this aspect may include one or more of the following optional features: in some embodiments, the method further comprises concentrating the cells prior to lysing the cells; in some embodiments, the incubation container contains from about 1 ml to about 50 ml of enrichment media (e.g., broth such as neutralizing buffer); in some embodiments, the enrichment media is selective for growth of Listeria; in some embodiments, the enrichment media is non-selective for growth of Listeria; in some embodiments, the method comprises incubating the cells in the incubation container for from about 4 to about 12 hours; in some embodiments, the collection device is a swab, q-tip, or sponge; in some embodiments, the collection device is a sponge; in some embodiments, the time from collection of the sample to detection of Listeria nucleic acids is 12 hours or less; in some embodiments, the time from collection of the sample to detection of Listeria nucleic acids is 8 hours or less; in some embodiments, the detection assay is an isothermal nicking enzyme amplification reaction (NEAR) assay; in some embodiments, the detection assay is a thermostable helicase-dependent isothermal amplification (tHDA) assay; in some embodiments, the detection assay is a molecular method such as Real time polymerase chain reaction (qPCR); in some embodiments, the method further comprises concentrating the incubated cells by centrifuging or filtering; in some embodiments, the nucleic acid sequences targeted by the detection assay are from RNA released from the cells; in some embodiments, the environmental sample is collected from a low-concentration environment comprising from about 2 to about 50 cells of Listeria or from about 5 to about 50 cells of Listeria; in some embodiments, the environmental sample, prior to incubation, comprises from about 2 to about 50 cells or from about 5 to about 50 cells of Listeria; in some embodiments, the method is of sufficient sensitivity to detect the presence of Listeria in an environmental sample having as little as 7 cells or as little as 3 cells of Listeria; in some embodiments, the collection device is a sponge and the incubation container contains from about 5 ml to about 50 ml of enrichment media (e.g., broth); in some embodiments, the collection device is a swab and the incubation container contains from about 1 ml to about 10 ml of enrichment media (e.g., broth).

In another aspect, provided herein is a kit for detecting the presence of Listeria in an environmental sample. The kit includes a collection device for collecting an environmental sample comprising cells; an incubation container containing about 50 ml or less of enrichment media; and optionally an assay for amplification and detection of Listeria nucleic acids in the sample.

Embodiments of this aspect may include one or more of the following optional features: in some embodiments, the kit is capable of detecting Listeria from a low-concentration environment in a time from collection of the sample to detection of the amplified nucleic acids of 12 hours or less; in some embodiments, the incubation container contains from about 1 ml to about 50 ml of enrichment media; in some embodiments, the enrichment media is selective for enrichment of Listeria; in some embodiments, the enrichment media is LESS Plus enrichment media; in some embodiments, the collection device is a swab, q-tip, or sponge; in some embodiments, the assay is a thermostable helicase-dependent isothermal amplification (tHDA) assay; in some embodiments, the assay is an isothermal nicking enzyme amplification reaction (NEAR) assay.

### BRIEF DESCRIPTION OF THE DRAWINGS

These and other features, aspects, and advantages of the present invention may be better understood when the following detailed description is read with reference to the accompanying drawings.

FIG. 1 shows an exemplary workflow diagram for a method of detecting Listeria from an environmental sample.

### DETAILED DESCRIPTION

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicants intend to reflect individually each possible number that such a range could reasonably encompass, Applicants also intend for the disclosure of a range to reflect, and be interchangeable with, disclosing any and all sub-ranges and combinations of sub-ranges encompassed therein.

The terms “comprises” and variations thereof do not have a limiting meaning where these terms appear in the description and claims.

As used herein, “a,” “an,” “the,” “at least one,” and “one or more” are used interchangeably. Thus, for example, a microorganism can be interpreted to mean “one or more” microorganisms.

The term “and/or” means one or all of the listed elements or a combination of any two or more of the listed elements.

As used herein, the term “Listeria” refers to Gram-positive rod shaped bacteria, including the historically recognized species L. grayi, L. innocua, L. ivanovii, L. monocytogenes, L. seeligeri, and L. welshimeri, as well as the newly described species L. aquatica, L. booriae, L. cornellensis, L. fleischmannii, L. floridensis, L. grandensis, L. marthii, L. newyorkensis, L. riparia, and L. rocourtiae.

### Collection

Providing a sample to be tested may comprise providing a sample that is suspected of containing a target microorganism. The sample can be any sample that may include a target microorganism as defined herein. The present methods include the collection of an environmental sample (e.g., from a surface with a swab/sponge, or from soil, sediments, fomites). In the food/beverage safety industry, environmental sampling comprises collecting samples from the environment where foods/beverages are produced (e.g., a manufacturing plant or commercial kitchen) to determine whether the environment contains harmful bacteria, such as Salmonella or Listeria. The environment where foods/beverages are produced include food/beverage contact surfaces (e.g., slicers, mixers, utensils, or conveyors) and non-food contact surfaces (e.g., floors, drains, carts or equipment housing). Non-limiting examples of food- or beverage-processing environmental samples include food-handling surface samples (e.g., conveyor belts, blades, cutting surfaces, mixing equipment surfaces, filters, storage containers), room samples (e.g., walls, floors, drains, ventilation equipment), and cleaning equipment (e.g., hoses, cleaning tools). Environmental sampling is important because environmental contamination—absent proper monitoring and controls—may lead to contamination of finished food/beverage product.

In some embodiments, the presence or absence of a target microorganism can be analyzed in a test sample that is derived from a variety of food or beverage sources. Non-limiting examples of food sources include raw or processed meat, raw or processed fruits or vegetables, non-fluid dairy products (e.g., cheese, butter, and ice cream), nuts, spices, ingredients, and syrups. Non-limiting examples of beverage sources include potable water, fruit or vegetable juices, milk, and fermented beverages. Pasteurized food or beverages may also be suitable sources.

In some embodiments, the environmental sample is from a low-concentration environment, i.e., an environment (e.g., a surface) with a low concentration of Listeria cells. As used herein, the term “low-concentration environment” means from about 1 to about 50 cells of Listeria in the sample collected from the environment. In other embodiments, the concentration of Listeria in the sample is from about 2-50 cells, 5-50 cells, 5-25 cells, less than or equal to 50 cells, less than or equal to 40 cells, less than or equal to 30 cells, less than or equal to 20 cells, less than or equal to 10 cells or less than or equal to 5 cells. In some embodiments, the concentration of Listeria in the sample is from about 1-50 CFU, 2-50 CFU, 5-50 CFU, 5-25 CFU, less than or equal to 50 CFU, less than or equal to 40 CFU, less than or equal to 30 CFU, less than or equal to 20 CFU, less than or equal to 10 CFU, or less than or equal to 5 CFU. As used herein, the term “CFU” means a colony forming unit, which is a unit used in microbiology to estimate the number of viable bacteria (or fungal) cells in a sample. In other embodiments, the environmental sample is from an environment such that the greater than 50 cells and/or greater than 50 CFU of Listeria are present in the sample. In some embodiments, the environment is such that from about 1-100 cells or greater and/or about 1-100 CFU or greater of Listeria are present in the environmental sample.

Methods and kits of the present invention may include a collection device. The collection device collects an environmental sample from a surface.

In some embodiments, the collection device is a sponge, e.g., an environmental sponge. For example, the EZ Reach Sponge Sampler with polyurethane sponge (World Bioproducts LLC) can be used. In some embodiments, the sponge is pre-moistened in broth, e.g. Letheen broth, LESS Plus broth (Neogen Corporation), LESS broth, or Demi-Fraser broth. In some embodiments, the sponge is pre-moistened in neutralizing buffer, buffered peptone water, or culture medium. In some embodiments, the volume of the pre-moistening liquid on the sponge is about 5-25 ml. A sponge advantageously samples a larger surface area than other sampling devices.

In some embodiments, the collection device is a swab. The swab may be comprised of cotton or polyester and may be pre-moistened in a broth solution. In some embodiments, the swab is pre-moistened in Letheen broth. In other embodiments, the swab is pre-moistened in Neutralizing Buffer, Buffered Peptone Water, or culture medium. In some embodiments, the volume of the pre-moistening liquid broth on the swab is about 1-10 ml. In some embodiments, the swab is provided as a polypropylene tube and cap containing a Letheen broth solution and a swab with polypropylene shaft and polyester fiber tip. In some embodiments, the swab is sold as Veriswab Samplers with Letheen Broth (World Bioproducts LLC). In some embodiments, the swab is sold as 3M Swab-Sampler with Letheen Broth. In some embodiments, the swab is sold as 3M Quick Swab.

In some embodiments, collecting an environmental sample comprises swabbing, with the collection device (e.g., swab or sponge) an area of a surface. In some embodiments, the area is a 1×1 inch area, a 4×4 inch area or a 12×12 inch area. In some embodiments, swabbing comprises swabbing in multiple directions. In some embodiments, the surface is a flat surface. In some embodiments, the environmental sample collected comprises from about 5 to about 50 cells or from about 2 to about 50 cells of Listeria. In some embodiments, the environmental sample collected comprises from about 5 to about 50 CFU or from about 2 to about 50 CFU of Listeria. In some embodiments, the environmental sample collected comprises greater than 50 cells or greater than 50 CFU of Listeria.

### Incubation

Upon collection of an environmental sample, the sample undergoes incubation to increase the number of cells (and thus the number of target nucleic acids) in the sample that are available for detection. —The relative proportion of live cells to dead cells in the sample will have shifted, in some instances significantly, to a larger proportion of live cells. This is advantageous in food safety testing, where the presence of live cells on the environmental surfaces is of particular concern.

Incubation is performed in an incubation container. The incubation container may be a bag, bottle, or other suitable container. In some embodiments, the incubation container is a bag, e.g., a Whirl-Pak bag. In some embodiments, the incubation container is equipped with a shaker device, i.e., a shaker incubator.

The incubation container holds the enrichment media/broth with the environmental sample. The environmental sample may be transferred to the incubation container by placing the collection device (e.g., sponge or swab) directly into the incubation container so that the collection device is contacting the broth. The enrichment media/broth may be selective for enrichment of Listeria. For example, the enrichment broth may be Letheen broth, LESS Plus broth (Neogen Corporation), LESS broth, or Demi-Fraser broth. In some embodiments, the enrichment broth comprises one or more (e.g., most or all) of the following components: enzymatic digest of casein, enzymatic digest of soybean meal, yeast extract, dextrose, sodium chloride, monopotassium phosphate, dipotassium phosphate, disodium phosphate, cycloheximide, nalidixic acid, acriflavine, and water. In some embodiments, the enrichment broth comprises one or more of 3-morpholinopropapanesulfonic acid, lithium chloride, and sodium carbonate. In some embodiments, the enrichment broth comprises one or more (e.g., most or all) of the following components: trypticase soy broth, yeast extract, monopotassium phosphate, disodium phosphate, sodium pyruvate, acriflavine HCl, nalidixic acid, cycloheximide, and water.

A low volume of enrichment broth is used, i.e., about 50 ml or less (e.g., about 1 ml to about 50 ml). In some embodiments, the volume of enrichment broth in the incubation container is from about 1 ml to about 50 ml, e.g., about 5 ml, about 10 ml, about 25 ml, or about 50 ml. In some embodiments, the volume of enrichment broth in the incubation container is from about 5 ml to about 50 ml. In some embodiments, the volume of enrichment broth in the incubation container is from about 1 ml to about 10 ml. In some embodiments, when the collection device is a sponge, about 5 ml to about 50 ml, about 5 ml to about 25 ml, or about 25 ml to about 50 ml (e.g., about 25 ml) of enrichment broth is used. In some embodiments, when the collection device is a swab, about 1 ml to about 10 ml, about 1 to about 5 ml, or about 5 ml to about 10 ml (e.g., about 5 ml) of enrichment broth is used. The low volume of enrichment broth results in higher concentration of the test analyte, which aids in the detection of Listeria with high sensitivity (low limit of detection (LOD)) with a shorter enrichment period.

The sample in the enrichment broth is incubated in the incubation container for an incubation period of about 12 hours or less, e.g., from about 4 to about 12 hours, or from about 6 to about 12 hours. In some embodiments, the incubation period is from about 4-10 hours, from about 4-8 hours, from about 6-10 hours, or from about 6-8 hours. In some embodiments, the incubation period is from about 7-10 hours, or from about 7-9 hours. In some embodiments, the incubation is performed at a temperature of about 36° C.

In some embodiments, a portion of the environmental sample (e.g., of about 1 ml or 5 ml) is incubated for longer time (up to 24 h) and is used for a culture confirmation method. The culture confirmation method may comprise obtaining a portion of the sample (e.g., 1 ml or 5 ml) and combining the portion with additional broth (e.g., LESS Plus enrichment broth or Listeria indicator broth) and streaking onto a culture medium (e.g., selective chromogenic agar).

### Sample Lysis

In some embodiments, the incubated sample undergoes concentration to increase the concentration of cells per volume of liquid. Suitable concentration methods include filtration, centrifugation, and use of magnetic beads. Concentrated cells where liquid media/broth (supernatant) has been removed may be resuspended in a smaller volume of liquid, e.g., a lysis buffer.

In some embodiments, an aliquot (50 to 250 μL) of the incubated sample is added directly into the lysis buffer, without the need for any further concentration steps.

The sample, after incubation and or concentration, undergoes lysis to release nucleic acids detectable by a Listeria detection assay. Lysis may be by suitable methods such as thermal lysis, chemical lysis, mechanical lysis, etc. In some embodiments, the cells are suspended in a lysis buffer comprising lytic enzymes that lyse the cells. In some embodiments, the cells are suspended in a lysis buffer and remain in the buffer during the detection assay. For example, the cells may be suspended in ANSR® lysis buffer (Neogen Corporation) comprising sodium sulfate, magnesium sulfate, poly(ethylene oxide)octylphenyl ether, potassium phosphate, lysozyme, and proteinase K.

### Detection

The nucleic acids released upon lysis are detected in an assay that detects the presence of Listeria. In some embodiments, the assay utilizes DNA amplification. In some embodiments, the assay uses a reverse transcriptase to produce cDNA from a target RNA. In some embodiments, the assay is an isothermal nicking enzyme amplification reaction (NEAR) assay. In some embodiments, the assay is a thermostable helicase-dependent isothermal amplification (tHDA) assay. In some embodiments, the assay utilizes fluorescence detection. In some embodiments, the assay is a quantitative polymerase chain reaction (qPCR) assay. In some embodiments, the assay is a loop-mediated isothermal amplification (LAMP) assay, a nucleic acid sequence-based amplification (NASBA) assay, a strand displacement amplification (SDA) assay, a multiple displacement amplification (MDA) assay, a rolling circle amplification (RCA) assay, or a ramification amplification method (RAM) assay.

In some embodiments, the assay provides results in less than about 4 hours, e.g., about 2 hours or less, or about 1 hour or less.

In some embodiments, the entire detection process, from sample collection to results of detection assay is completed in about 12 hours or less, e.g., about 10 hours or less, about 9 hours or less, about 8 hours or less, about 7 hours or less, or about 6 hours or less. In some embodiments, the entire detection process, from sample collection to results of detection assay is completed in about 6 to about 12 hours, e.g., about 6-10 hours, about 8-10 hours, about 7-9 hours, or about 6-8 hours.

Methods and reagents for isothermal detection and amplification of nucleic acids (e.g., RNA) are described in International Patent Application Publication No. WO 2009/012246, which is incorporated by reference herein in its entirety. Briefly, the methods of amplifying nucleic acid target sequences rely on nicking and extension reactions to amplify shorter sequences in a shorter timeframe than traditional amplification reactions, such as, for example, strand displacement amplification reactions. Embodiments of the invention include, for example, reactions that use only two templates to amplify a target sequence, one or two nicking enzymes, and a polymerase, under isothermal conditions. In exemplary embodiments, the polymerase and the nicking enzyme are thermophilic, and the reaction temperature is significantly below the melting temperature of the hybridized target region. The nicking enzyme nicks only one strand in a double-stranded duplex, so that incorporation of modified nucleotides is not necessary as in the case of conventional strand displacement amplification. An initial heat denaturation step is not required for the methods of the present invention. Due to the simplicity of the reaction, in exemplary embodiments, the reaction is very easy to perform, requires no special equipment, such as a thermocycler, and can amplify 20-30mer products 108 to ˜1000 fold from genomic DNA in only about 2.5 to about 10 minutes. The method is able to amplify RNA during a simultaneous reverse transcription step.

The ANSR® Listeria assay is an isothermal amplification and detection system that targets ribosomal RNA (rRNA), a high copy number target. Lysis of even a single Listeria cell can release on the order of 1,000 to 10,000 copies of rRNA. Since the lysis reaction is performed in a small volume, target concentration is sufficiently high that a 5-100 μL aliquot (e.g. 50 microliters) of the lysate subsequently transferred to the ANSR® assay reagent tube will contain a sufficient number of target rRNA molecules for detection.

Further methods for isothermal detection and amplification of nucleic acids are described in U.S. Pat. No. 7,662,594, which is incorporated by reference herein in its entirety. These methods describe helicase-dependent amplification of nucleic acids (e.g., RNA). Embodiments of the invention include, for example, adding to Listeria RNA a mixture comprising reverse transcriptase, a helicase (e.g., thermostable UVrD helicase), optionally a single stranded binding protein, oligonucleotide primers and one or more polymerases; and reverse transcribing the RNA to form cDNA and amplifying, by helicase-dependent amplification, the cDNA.

In some embodiments, the assay result is read by an automated reader. In other embodiments, the assay result is detected by enzymatic detection methods or gel electrophoresis.

In some embodiments of the inventive method, the assay detects target nucleic acids released from the Listeria cells in a low-concentration environment. The (lower) limit of detection (LOD) of the assay may be about 7 cells (i.e. 7 cells of Listeria in the environmental sample) or the LOD may be about 5 cells, or the LOD may be about 3 cells or the LOD may be about 2 cells. In other embodiments, the LOD of the assay may be such that the assay is able to detect as little as 1-10 cells, 1-5 cells, 2-5 cells, 5-10 cells, 5-15 cells, or 10-20 cells, e.g., as little as 1, 2, 3, 4, 5, 6, 7, 8, 9, 10, 11, 12, 13, 14, 15, 16, 17, 18, 19 or 20 cells of Listeria in the sample collected from the environment. In some embodiments, the LOD of the assay may be about 7 CFU (i.e. 7 CFU of Listeria in the environmental sample) or the LOD may be about 5 CFU, or the LOD may be about 3 CFU, or the LOD may be about 2 CFU. In other embodiments, the LOD of the assay may be such that the assay is able to detect as little as 1-10 CFU, 1-5 CFU, 2-5 CFU, 5-10 CFU, 5-15 CFU, or 10-20 CFU, e.g., as little as 1, 2, 3, 4, 5, 6, 7, 8, 9, 10, 11, 12, 13, 14, 15, 16, 17, 18, 19 or 20 CFU of Listeria in the sample collected from the environment.

### Kits

Aspects of the invention include kits with components used in the methods described herein. Kits may include one or more of: a collection device, a collection device with a broth, an incubation container, enrichment media, an incubator, concentration equipment and supplies (a centrifuge, centrifuge tubes, a filter, magnetic beads), lysis buffer, and assay equipment and reagents (primers, probes, dNTPs, enzymes, optical readers, etc.).

In some embodiments, a kit for detecting the presence of Listeria in an environmental sample comprising: a collection device for collecting an environmental sample comprising cells; an incubation container containing about 50 ml or less of enrichment media; and an assay for amplification and detection of Listeria nucleic acids in the sample.

In some embodiments, the incubation container contains from about 1 ml to about 50 ml of enrichment media. In some embodiments, the enrichment media is selective for enrichment of Listeria. In some embodiments, the enrichment media is LESS Plus enrichment media. In some embodiments, the collection device is a swab, q-tip, or sponge. In some embodiments, the assay is a thermostable helicase-dependent isothermal amplification (tHDA) assay. In some embodiments, the assay is an isothermal nicking enzyme amplification reaction (NEAR) assay.

### EXAMPLES

### Example 1

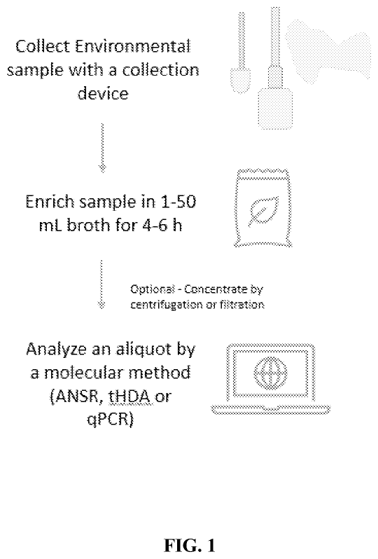

An exemplary process for detecting Listeria from an environmental sample, such as shown in FIG. 1, is as follows:

1) Wipe surface with an environmental sponge or a swab to collect environmental sample.

2) Place sponge in a Whirl-Pak® bag containing 2-25 ml of LESS Plus enrichment broth or a non-selective broth.

3) Incubate the sample at 35° C. for about 4-6 hours. (Set aside 1 ml of sample in enrichment broth for culture confirmation)

4) Concentrate the cells in the sample if desired by centrifugation and removal of supernatant.

5) Resuspend the cells in a small volume of lysis buffer or take an aliquot of enriched broth and lyse the cells.

6) Run the sample (or an aliquot thereof) in an ANSR® (NEAR) or tHDA assay (about 1 hour) to detect Listeria.

The total time from sample collection to Listeria detection is about 6-8 hours.

In parallel, an exemplary culture confirmation method can be used to verify the reliability of detection:

1) Obtain 1 ml of the sample in the enrichment broth.

2) Add 9-10 mL of LESS Plus enrichment broth or Listeria indicator broth. Incubate the sample at 35° C. for about 16-24 hours.

3) Streak onto selective/chromogenic agar.

### Example 2

Listeria Same Day Environmental Sample Study

The following materials were used:
L. monocytogenes ATCC 19115,
ANSR Listeria assay kits,
Environmental swabs and sponges premoistened with Letheen neutralizing buffer,
Phosphate Buffered Saline (PBS), Tryptic Soy Broth (TSB), LESS Plus
Enrichment Broth, Modified Agar (MOX).

Environmental Sample Preparation:

Environmental swabs (Letheen swabs) and sponges (EZ sponges) were used to wipe (1×1 inch surface using swabs and 4×4 inch surface using sponges) the environmental surfaces (sink drain, floor drain) at a manufacturing facility, so that the swabs and sponges were contaminated with the natural microflora. The contaminated swabs/sponges were inoculated with low levels of L. monocytogenes (2-7 CFU) and held in the refrigerator for 3 days to simulate the recommended cold stressing procedure for Listeria cells.

Listeria Same Day Procedure:

After 3 days holding period at 4° C. for simulating cold stress, 10 mL (for swabs) or 25 mL (for sponges) of LESS Plus enrichment broth was added to the swabs or sponges, mixed thoroughly to release and extract the target organisms into the broth. The samples were incubated for 6 hours at 35° C. At the end of the enrichment period, an aliquot (1 mL) was taken for centrifugation at 5000 g for 10 min. The supernatant was removed, and 500 μL of ANSR lysis buffer was added and taken through the lysis step. For samples tested with no concentration step, at the end of the enrichment period, an aliquot (50 μL) was removed, and 450 μL of ANSR lysis buffer was added for the lysis step.

ANSR Listeria Procedure:

The sample was then tested by the isothermal ANSR Listeria assay. Briefly, the sample was lysed at 37° C. for 10 min, followed by the heat inactivation step at 80° C. for 20 min. An aliquot (50 μL) of the lysate was added to rehydrate the LRN reagent for detection in the ANSR reader. In Table 1, positive ANSR Listeria results were noted as the total detected over total samples tested (#pos/#test), indicating the presence or absence of Listeria in the environmental sample.

Culture Confirmation Procedure:

Continued incubating the remaining swab and sponge samples with broth at 35° C. for total of 24 h followed by streaking on MOX plate for observing and counting typical Listeria colonies. In Table 1, positive culture results were noted as the total detected over total samples tested (#pos/#test), indicating the presence or absence of Listeria in the environmental sample.

Results and Conclusions:

Table 1 shows the results of the study.

The Listeria Same Day assay with 6 hour enrichment (with or without concentration via centrifugation step) detected low levels (2-7 CFU) of Listeria effectively using swabs and sponges with the presence of the background microflora. The assay showed comparable results with the culture method that required enrichment of samples for 24 hours. Therefore, it has surprisingly been discovered that the Listeria Same Day assay of this Example 2 can be used to detect Listeria from environmental surfaces, including low-concentration environments, within the same day or same work shift of sample collection.

TABLE 1

| | | | ANSR | ANSR | Culture |
|---|---|---|---|---|---|
| Sampler | Sample | Inoculum | Listeria | (centrifuged) | confirmation |
| type | area | Lm (CFU) | #pos/#test | #pos/#test | #pos/#test |
| Swabs | Floor | 2.2 | 4/4 | 4/4 | 4/4 |
| | Drain | 6.5 | 4/4 | 4/4 | 4/4 |
| | Sink | 2.2 | 4/4 | 4/4 | 4/4 |
| | Drain | 6.5 | 4/4 | 4/4 | 4/4 |
| Sponges | Floor | 2.2 | 3/4 | 4/4 | 3/4 |
| | Drain | 6.5 | 4/4 | 4/4 | 4/4 |
| | Sink | 2.2 | 4/4 | 4/4 | 4/4 |
| | Drain | 6.5 | 4/4 | 4/4 | 4/4 |

What is claimed is:

1. A method of detecting Listeria in an environmental sample, the method comprising the steps of:
collecting an environmental sample comprising cells using a collection device;
transferring the sample to an incubation container containing about 50 ml or less of enrichment media;
incubating the cells in the incubation container for about 12 hours or less;
lysing the cells to release nucleic acids from the cells; and
performing a detection assay that targets nucleic acid sequences for amplification and detection of Listeria nucleic acids released from the cells.

2. The method of claim 1, further comprising concentrating the cells prior to lysing the cells.

3. The method of claim 1, wherein the incubation container contains from about 1 ml to about 50 ml of enrichment media.

4. The method of claim 1, wherein the enrichment media is selective for growth of Listeria.

5. The method of claim 1, wherein the enrichment media is non-selective for growth of Listeria.

6. The method of claim 1, wherein the collection device is a swab, q-tip, or sponge.

7. The method of claim 6, wherein the collection device is a sponge.

8. The method of claim 1, wherein the time from collection of the sample to detection of the amplified nucleic acids is 12 hours or less.

9. The method of claim 8, wherein the time from collection of the sample to detection of the amplified nucleic acids is 8 hours or less.

10. The method of claim 1, wherein the detection assay is a thermostable helicase-dependent isothermal amplification (tHDA) assay.

11. The method of claim 1, wherein the detection assay is an isothermal nicking enzyme amplification reaction (NEAR) assay.

12. The method of claim 2, wherein concentrating the incubated cells comprises centrifuging or filtering.

13. The method of claim 1, wherein the environmental sample is collected from a low-concentration environment comprising from about 2 to about 50 cells of Listeria, and wherein the detection assay detects Listeria in the sample.

14. The method of claim 13, wherein the low-concentration environment comprises from about 5 to about 50 cells of Listeria.

15. The method of claim 1, wherein the environmental sample, prior to incubation, comprises from about 2 to about 50 cells of Listeria, and wherein the detection assay detects Listeria in the sample.

16. The method of claim 15, wherein the environmental sample, prior to incubation, comprises from about 5 to about 50 cells of Listeria.

17. The method of claim 1, wherein the nucleic acid sequences targeted by the detection assay are from RNA released from the cells.

18. The method of claim 1, wherein the method is of sufficient sensitivity to detect the presence of Listeria in an environmental sample having as little as 7 cells of Listeria.

19. The method of claim 1, wherein the method is of sufficient sensitivity to detect the presence of Listeria in an environmental sample having as little as 3 cells of Listeria.

20. The method of claim 3, wherein the collection device is a sponge and the incubation container contains from about 5 ml to about 50 ml of enrichment media.

21. The method of claim 3, wherein the collection device is a swab and the incubation container contains from about 1 ml to about 10 ml of enrichment media.

22. A kit for detecting the presence of Listeria in an environmental sample comprising:
a collection device for collecting an environmental sample comprising cells;
an incubation container containing about 50 ml or less of enrichment media; and
an assay for amplification and detection of Listeria nucleic acids in the sample;
wherein the kit is capable of detecting Listeria from a low-concentration environment in a time from collection of the sample to detection of the amplified nucleic acids of 12 hours or less.

23. The kit of claim 22, wherein the incubation container contains from about 1 ml to about 50 ml of enrichment media.

24. The kit of claim 22, wherein the enrichment media is LESS Plus enrichment media.

25. The kit of claim 22, wherein the collection device is a swab, q-tip, or sponge.

26. The kit of claim 22, wherein the assay is a thermostable helicase-dependent isothermal amplification (tHDA) assay or an isothermal nicking enzyme amplification reaction (NEAR) assay.

27. The method of claim 1, comprising incubating the cells in the incubation container for from about 4 to about 12 hours.
